(19) United States
(12) Patent Application Publication
(10) Pub. No.: US 2026/0224712 A1
(43) Pub. Date: Aug. 6, 2026

(54) GLYCOCONJUGATES FOR PHARMACEUTICAL USE

(71) Applicant: Hiroshi MURAKAMI, Morioka Iwate (JP)

(72) Inventor: Hiroshi MURAKAMI, Morioka Iwate (JP)

(21) Appl. No.: 19/205,973

(22) Filed: May 12, 2025

(30) Foreign Application Priority Data
Feb. 2, 2025 (JP) 2025-015752

(51) Int. Cl. A61K 47/54

(52) U.S. Cl. CPC A61K 47/549

(57) ABSTRACT

Disclosed are glycoconjugates for pharmaceutical use that are safe for the human body. The glycoconjugates for pharmaceutical use comprise a first monosaccharide having a non-reducing terminal at one end, two or more second monosaccharides attached to the first monosaccharide via a glycosidic bond, and at least one amino acid attached to a reducing terminal of one of the second monosaccharides at the other end via an N or O bond. The glycoconjugates adhere to lectins of human tissue that an infection source targets for adhesion, but not to selectins of the human tissue.

### CROSS-REFERENCE TO RELATED APPLICATION(S)

This application is based upon and claims the benefit of priority from Japanese Patent Application No. 2025-015752, filed Feb. 2, 2025, the entire contents of which are incorporated herein by reference.

### TECHNICAL FIELD

Embodiments relate to glycoconjugates for pharmaceutical use.

### BACKGROUND

Infectious diseases caused by bacteria and viruses can lead to global outbreaks and, depending on the pathogen, cause numerous deaths. Recently, COVID-19 and HIV are fresh in our minds. When the spread of infection begins, vaccines and other measures are developed. However, not only do vaccines take time to establish acquired immunity, but vaccine development cannot keep pace with rapid changes in the envelope protein of the virus.

In this regard, it has been proposed to administer mannose, based on the finding that pathogens use lectins to identify adhesion sites within the human body (e.g., International publication WO 2005/023289). However, this technology does not consider the adverse effects of mannose on the immune system, etc., even if mannose inhibits adhesion between lectins and viruses.

Specifically, for example, when a human cell is infected with an infectious agent, it releases P-selectin stored inside the cell from the cell membrane surface into the blood vessels, and leukocytes leach into the damaged cell by binding to the leukocyte's sugar chain ligand, which specifically binds to this P-selectin leukocytes leach into the damaged cells (see, for example, Harvey Lodish et al., Molecular Cell Biology, 9th Edition, Jul. 27, 2023, Tokyo Kagaku Doujin Co., page 830; and Maureen E. Taylor et al., Introduction to Glycobiology, Nov. 1, 2005, Kagakudojin, Inc., page 138).

Thus, when administered mannose binds to P-selectin, leukocytes are unable to identify the damaged cells, causing new damage to the human body.

### SUMMARY

The above indicates the occasion (trigger) that led to the embodiments, and do not limit the technical scope of the embodiments, nor do they allow for a limited interpretation of the technical scope of the embodiments.

Embodiments provide glycoconjugates for pharmaceutical use comprising a first monosaccharide having a non-reducing terminal at one end, two or more second monosaccharides attached to the first monosaccharide via a glycosidic bond, and at least one amino acid attached to a reducing terminal of one of the second monosaccharides at the other end via an N or O bond. The glycoconjugates adhere to lectins of human tissue that an infection source targets for adhesion, but not to selectins of the human tissue.

With the embodiments, it is possible to provide pharmaceutical glycoconjugates safe for the human body.

### BRIEF DESCRIPTION OF THE DRAWINGS

FIG. 1 are examples of monosaccharides contained in ordinary sugar chains.

FIG. 2 illustrates bonding of the terminal portion of a sugar chain.

FIG. 3 is a diagram illustrating an example of the invention.

### DETAILED DESCRIPTION

The following is a detailed description of one embodiment of pharmaceutical glycoconjugates with reference to the drawings.

### Basic Concept

Pathogens such as viruses adhere to cells at specific sites in the human body and establish infection by entering those cells. This is because pathogens possess glycan ligands that specifically bind to specific lectins in the human body.

For example, when HIV enters the human body, it first binds to DC-SIGN (dendritic cell-specific ICAM-3-grabbing non-integrin), a C-type lectin on the cell membrane surface of dendritic cells. Dendritic cells remain bound to HIV and approach T cells. HIV then presents and binds the CD4 glycan ligand possessed by the HIV envelope to the glycan receptor CD4 on the surface of the T cell and enters the T cell (see, for example, Maureen E. Taylor et al., Introduction to Glycobiology, Nov. 1, 2005, Kagakudojin, Inc., pages 143-144).

Here, even if the envelope protein changes, the lectins on the cell surface of the human body do not change. Therefore, it would require less development effort to block the lectins in the human body with glycans rather than to follow the changes in envelope proteins.

Therefore, embodiments provide a glycoconjugate compound for pharmaceutical use that contains a sugar chain with three or more monosaccharides attached, which do not bind to selectins of human tissue, but binds to lectins of human tissue targeted for adhesion by an infection source. Several types of selectins, such as P-selectin, E-selectin, and L-selectin, have been found at present. The pharmaceutical glycoconjugates of the embodiments do not bind to at least P-selectin, and the number of types of selectins to which the pharmaceutical glycoconjugates of the embodiments do not bind may be increased further.

### Structure of a Sugar Chain

A glycan chain is a portion of a monosaccharide that is continuously bound to multiple glycoproteins or glycolipids possessed by living organisms, including humans. There are 10 main types of monosaccharides known to be included in these glycans.

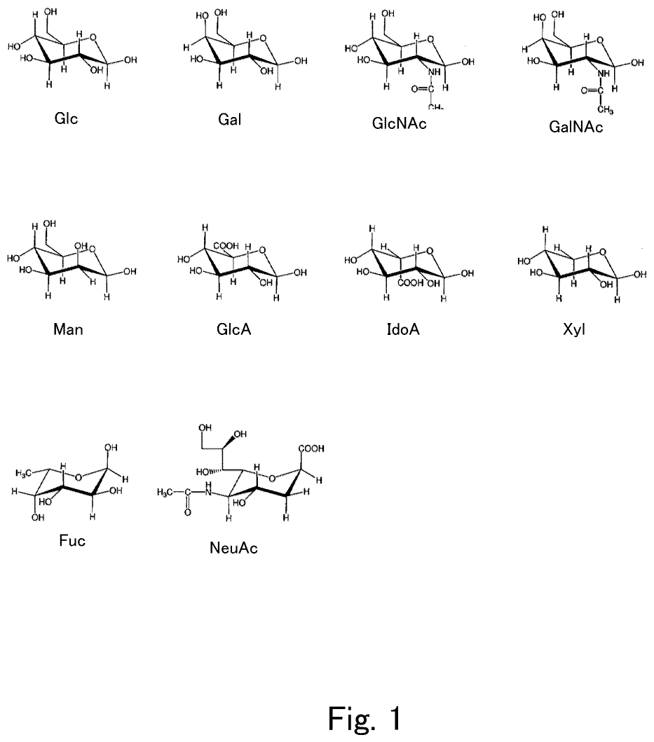

FIG. 1 shows examples of monosaccharides normally included in sugar chains. As shown in FIG. 1, monosaccharides normally included in sugar chains are glucose (Glc), galactose (Gal), N-acetylglucosamine (GlcNAc), N-acetylgalactosamine (GalNAc), mannose (Man), glucuronic acid (GlcA), iduronic acid (IdoA), xylose (Xyl), fucose (Fuc), and sialic acid (NeuAc).

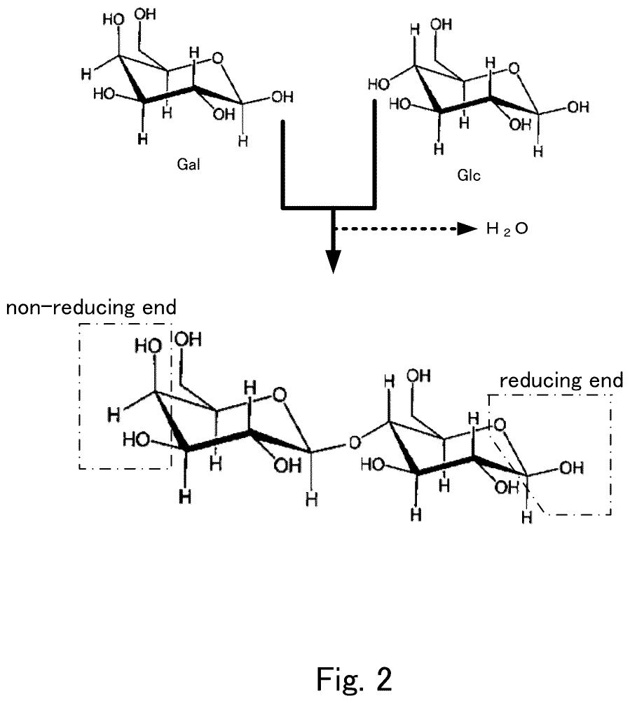

FIG. 2 shows how the terminal end of the sugar chain is bound. FIG. 2 illustrates galactose and glucose as examples. As shown in FIG. 2, galactose and glucose are joined by dehydration condensation. This bonding mode is called a glycosidic bond.

When two monosaccharides are joined by a glycosidic bond, a carboxyl group remains at one end. This part is called the reducing terminal. An alkyl group remains at the other end. This part is called the non-reducing terminal.

The reducing terminal may further bind to aspartic acid and other substances via nitrogen (N) or to serine and other substances via oxygen (O). The former is called an N bond and the latter an O bond.

### Glycoconjugates for Pharmaceuticals

This form of pharmaceutical glycoconjugate compound (hereinafter referred to as “short chain glycoconjugate”) has a monosaccharide with a non-reducing terminal at one end, two or more monosaccharides are attached to this monosaccharide via a glycosidic bond, and at least one amino acid is attached to the reducing terminal of the monosaccharide at the other end via an N bond or O bond.

The reason why the number of monosaccharides that short chain glycans have is set to three or more as described above is because the number of representative monosaccharides shown in FIG. 1 is 10, and therefore, when three monosaccharides are combined, the number of combinations is 1,000, which is sufficient to identify lectins and selectins in the human body. Therefore, if we want to identify lectins more strictly, we should increase the number of monosaccharides that short chain sugar chains have.

### Pharmaceuticals Containing Short-Chain Sugar Chains

There are no restrictions on pharmaceutical products containing short-chain sugar chains. For example, injectable solutions, tablets for oral administration, liquids for inhalation, powders for inhalation, etc., can be prepared as needed using known formulations. Since specific known formulations are readily available to those skilled in the art through various non-patent literature as well as patent literature, a detailed description is omitted here.

### Selection of Monosaccharide Combinations and Sequences of Short Chain Glycans

The combinations and sequences of monosaccharides of short chain glycans are selected from the combinations and sequences that adhere to lectins in human tissues targeted by the infection source, excluding combinations and sequences that adhere to selectins in the human body.

Since representative glycans that adhere to lectins and selectins are already in the database along with their adhesion strengths, the monosaccharide combinations and sequences can be selected by referring to this database. Examples of databases are listed in the following URLs:
Site Name: LfDB
URL: Https://acgg.asia/lfdb2/

Glycan ligands for lectins and selectins not listed in the above databases can be selected as follows.

### Step 1

Five types of monosaccharides are selected, and amino acids are attached to the reducing terminals of these monosaccharides by N or O bonds to prepare five types of short-chain sugar chains to make the first short-chain sugar chain. Hereafter, the short chain sugar chain is also referred to as the target short chain sugar chain.

### Step 2

The presence or absence of adhesion of target lectins and target selectins to the first short-chain glycans is examined. If it is known which of the five short chain glycans is bound to which monosaccharide, the bound monosaccharide is the monosaccharide at the non-reducing terminal of the target short chain glycan. If it is not known, perform the following (Step 3).

### (Step 3) (if There Were Short Chain Glycans to Attach)

In this case, the five monosaccharides selected will contain monosaccharides that adhere to the target lectin or target selectin. Therefore, these five monosaccharides are further divided into groups of three and two to examine the adhesion of target lectins and target selectins to the first short-chain glycans. This is repeated to determine the monosaccharide at the non-reducing terminal of the target short chain glycan.

### (Step 3) (if There Were No Short Chain Sugar Chains to Attach)

In this case, the remaining five monosaccharides are examined for adhesion in the same manner to determine the monosaccharide at the non-reducing terminal of the target short chain glycan.

### Step 4

To the monosaccharide at the non-reducing terminal of the determined target short-chain sugar chain, five types of monosaccharides are selected from the ten types of monosaccharides mentioned above and attached to the reducing terminal of this attached monosaccharide by an N bond or O bond to prepare five types of short chain sugar chains, which are used as the second short chain sugar chain.

### Step 5

Perform (step 2) to (step 3) for the second short chain glycan chain in the same manner to determine the next monosaccharide to be bound after the monosaccharide having the non-reducing terminal of the target short chain glycan chain.

### Step 6

Five types of monosaccharides are selected from the ten types of monosaccharides mentioned above to be bound to the monosaccharide next to the monosaccharide having the non-reducing terminal of the determined target short-chain sugar chain, and five types of short chain sugar chains are prepared by binding an amino acid by an N bond or O bond to the reducing terminal of this bound monosaccharide, and the third short chain sugar chain The third short-chain sugar chain and the third short-chain sugar chain.

### Step 7

Perform (step 2) to (step 3) for the third short-chain glycan chain in the same manner to determine the combination and sequence of monosaccharides that bind to the next to the monosaccharides having the non-reducing terminal of the target short-chain glycan chain.

The combination and sequence of monosaccharides of the target short-chain glycans that do not adhere to selectins in the human body can be determined in the same way.

### Preparation of Target Short-Chain Glycans

The monosaccharides are linked in sequence according to the order of monosaccharides determined above, and an amino acid is attached to the reducing terminal of the monosaccharide at the opposite end of the monosaccharide having the non-reducing terminal by an N bond or O bond to form the target short-chain glycan chain. Known methods of joining monosaccharides, N-bond, and O-bond can be used as appropriate. In other words, it is not necessary to prepare 1,000 different short chain glycans to determine how to sequence three monosaccharides.

The following literature (hereinafter referred to as DOC 1) describes examples of C-type lectins and P-selectins.
DOC 1: Biochemistry, 2018, Vol. 90, No. 5, p. 651-663, the entire contents of which are incorporated by reference herein.

### C-Type Lectin

As for C-type lectins, among the portions of the sugar chain divided into three from mannose (represented by a circle, •) illustrated on the right side of (c) in FIG. 2 of DOC 1, the bottom portion, it is stated that the lectin recognizes and specifically binds to the 1-3 arm of the part illustrated in the bottom figure. In this part, three mannoses are linearly bound.

### P-Selectin

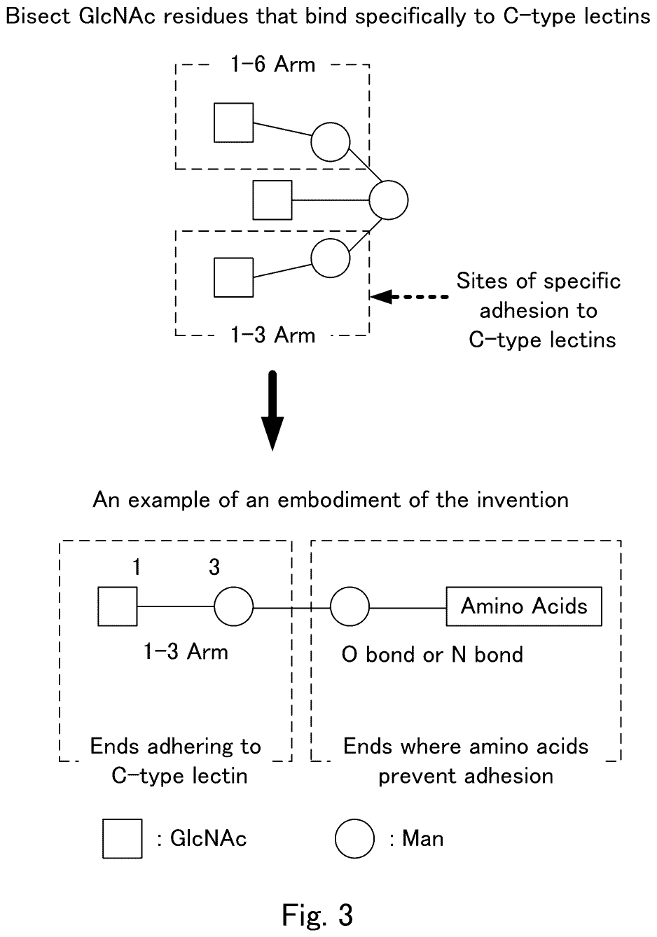

As for P-selectin, FIG. 3(b) of DOC 1 shows that it specifically binds to PSGL-1 peptide and sialyl Lewis-X bound by an O bond. P-selectin specifically binds to PSGL-1 peptide and sialyl Lewis-X bound by an O bond.

Here, the interaction between P-selectin and sialyl Lewis-X bound by an O bond is illustrated by three dashed lines in FIG. 3 of DOC1. Among the three dashed lines, the rightmost wavy line indicates that there is interaction with a part of the sugar chain. In other words, only fucose (Fuc, shown by the horizontal black triangle) bound to N-acetylglucosamine (GlcNAc, shown by the black square) of sialyl Lewis-X interacts.

### Embodiment 1

In one embodiment, the sugar chain that adheres to C-type selectin is one in which three mannoses are bound adjacent to each other, and at least one amino acid is bound to the reducing terminal of this monosaccharide, mannose, via an N or O bond by a known method.

There is no limitation to the amino acid to be bound since it only must adhere to the lectin and plug the adhesion site. Also, it should be recognized by a person having ordinary skill in the art that the above-described sugar chain of the embodiment consisting of three mannoses bound next to each other does not adhere to the above-mentioned P-selectin. Moreover, the above-described sugar chain of the embodiment can comprehensively prevent human infection with pathogens that adhere to the above-mentioned C-type lectin, regardless of the types and varieties of the pathogens.

FIG. 3 illustrates an example of the embodiments. One example of this embodiment is a glycoconjugate in which an amino acid is attached to the mannose (Man) at the opposite end of the 1-3 arm of bisecting GlcNAc residues by an O bond or N bond. The 1-3 arm of bisecting GlcNAc residues shown in FIG. 3 adheres specifically to C-type lectins. The reason for binding the amino acid to the mannose at the non-adhering end, which is opposite to the 1-3 arm of the bisecting GlcNAc residues, is to prevent the non-adhering end from adhering to selectins.

As described above, glycoconjugates for pharmaceutical use comprising; a first monosaccharide with a non-reducing terminal at one end; two or more second monosaccharides attached to this monosaccharide via a glycosidic bond; and at least one amino acid attached to a reducing terminal of one of the second monosaccharides at the other end via an N or O bond, wherein the glycoconjugates for pharmaceutical use adhere to lectins of human tissue that an infection source adheres to but do not adhere to selectins of the human tissue.

Glycoconjugates for pharmaceutical use, according to Embodiment 1 comprises: a first monosaccharide with a non-reducing terminal at one end; two or more second monosaccharides attached to this monosaccharide via a glycosidic bond; and at least one amino acid attached to a reducing terminal of one of the second monosaccharides at the other end via an N or O the other end via an N or O bond, wherein the glycoconjugates adhere to receptors of an infection source that an infection source uses for adhesion, but does not adhere to selectins of a monosaccharide via a glycosidic bond. not adhere to selectins of a human tissue.

Therefore, Embodiment 1 has the effect of providing the glycoconjugates for pharmaceutical use that are safe for the human body.

### Embodiment 2

In Embodiment 1, glycoconjugates for pharmaceutical use that adhere to lectins of human tissue were disclosed. In Embodiment 2, glycoconjugates for pharmaceutical use that blocks glycan receptors of infection sources that adhere to glycans on the surface of human or avian cell membranes are disclosed. Here, influenza A viruses (IAVs) are used as examples of infection sources.

It is already known that IAVs attach to the following sugar chains that extend externally from the surface of human or avian cell membranes:
(Neu5Ac)₂-Gal-GlcNAc-This

The sugar chain consists of two N-acetylneuraminic acid (Neu5Ac) molecules attached via galactose to N-acetylglucosamine (GlcNAc) on the cell membrane surface of the human or avian being infected. One is bound to the α2 carbon of N-Acetylneuraminic acid by an α2-6 bond with the 6 carbon of galactose, and the other is bond to the α2 carbon of N-Acetylneuraminic acid by an α2-3 bond with the 3 carbon of galactose.

The part comprising α2-6 bond of N-acetylneuraminic acid binds to the receptor on human cells, while the part comprising α2-3 bond of N-acetylneuraminic acid binds to the receptor on avian cells. See, for example, Nico J. Overeem et al., “A Dynamic, Supramolecular View on the Multivalent Interaction between Influenza Virus and Host Cell,” Small 2021, Vol. 17, pp. 2007214-1 to 2007214-13, the entire contents of which are incorporated by reference herein.

In embodiment 2, glycoconjugates for pharmaceutical use are generated by the following known techniques. For human:
(Neu5ac)-<α2-6 bond>-Gal-Glcnac-<O Bond or N Bond>-amino acid

For avian:
(Neu5ac)-<α2-3 bond>-Gal-Glcnac-<O Bond or N Bond>-amino acid

What is claimed is:

1. Glycoconjugates for pharmaceutical use comprising:
a first monosaccharide with a non-reducing terminal at one end;
two or more second monosaccharides attached to the first monosaccharide via a glycosidic bond; and
at least one amino acid attached to a reducing terminal of one of the second monosaccharides at the other end via an N or O bond,
wherein the glycoconjugates adhere to lectins of human tissue that an infection source targets for adhesion, but does not adhere to selectins of the human tissue.

2. Glycoconjugates for pharmaceutical use comprising:
a first monosaccharide with a non-reducing terminal at one end;
two or more second monosaccharides attached to the first monosaccharide via a glycosidic bond; and
at least one amino acid attached to a reducing terminal of one of the second monosaccharides at the other end via an N or O bond,
wherein the glycoconjugates adhere to receptors of an infection source that the infection source uses for adhesion, but does not adhere to selectins of human tissue.

3. Glycoconjugates for pharmaceutical use comprising:
a first monosaccharide with a non-reducing terminal at one end;
two or more second monosaccharides attached to the first monosaccharide via a glycosidic bond; and
at least one amino acid attached to a reducing terminal of one of the second monosaccharides at the other end via an N or O bond,
wherein the glycoconjugates adhere to receptors of an infection source that the infection source uses for adhesion, but does not adhere to selectins of avian tissue.
